(19) United States
(12) Patent Application Publication
(10) Pub. No.: US 2026/0256140 A1
(43) Pub. Date: Sep. 3, 2026

(54) BIPHENYL COMPOUNDS CONTAINING SUBSTITUTED SULFIDE (SULFOXIDE) GROUPS AND USES THEREOF

(71) Applicants: METISA BIOTECHNOLOGY CO., LTD, Nanning City (CN); SHENYANG UNIVERSITY OF CHEMICAL TECHNOLOGY, Shenyang City (CN)

(72) Inventors: Lixin ZHANG, Shenyang City (CN); Jing ZHANG, Shenyang City (CN); Yixing GAO, Shenyang City (CN); Zhuo KANG, Shenyang City (CN)

(21) Appl. No.: 19/646,271

(22) Filed: Apr. 13, 2026

Related U.S. Application Data
(62) Division of application No. 18/365,343, filed on Aug. 4, 2023.
Application No. 18/365,343 is a continuation of application No. PCT/CN2022/074223, filed on Jan. 27, 2022.

(30) Foreign Application Priority Data
Feb. 5, 2021 (CN) 202110157762.2

(51) Int. Cl. A01N 41/10; A01N 39/00; A01P 7/02; A01P 7/04; C07C 317/14; C07C 321/26; C07C 323/09; C07C 327/36

(52) U.S. Cl. CPC A01N 41/10; A01N 39/00; A01P 7/02; A01P 7/04; C07C 317/14; C07C 321/26; C07C 323/09; C07C 327/36

(57) ABSTRACT

The present invention belongs to the field of agricultural insecticides and acaricides. In particular, the present invention relates to a biphenyl compound containing substituted sulfide (sulfoxide) groups and a use thereof. The structure is shown in Formula I. The definitions of each substituent in the formula are described in the description. The compound of Formula I has excellent insecticidal and acaricidal activities, and can be used for preventing and treating various pests and pest mites.

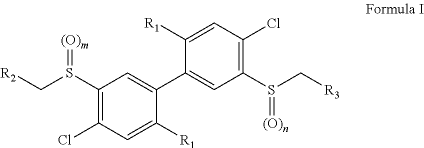

### RELATED APPLICATIONS

The present application is Divisional of U.S. application Ser. No. 18/365,343 filed Aug. 4, 2023, which is a Continuation of International Application Number PCT/CN2022/074223 filed Jan. 27, 2022, and claims priority to Chinese Application Number 202110157762.2, filed Feb. 5, 2021, the disclosure of which are hereby incorporated by reference herein in their entireties.

### TECHNICAL FIELD

The present invention belongs to the field of agricultural insecticides and acaricides, and particularly relates to a biphenyl compound containing substituted sulfide (sulfoxide) groups and a use thereof.

### BACKGROUND ART

Patent No. CN105541682A discloses a compound of the following formula, and specific compounds KC1 (compound No. 1), KC2 (compound No. 2), and KC3 (compound No. 3), which have certain acaricidal activity:

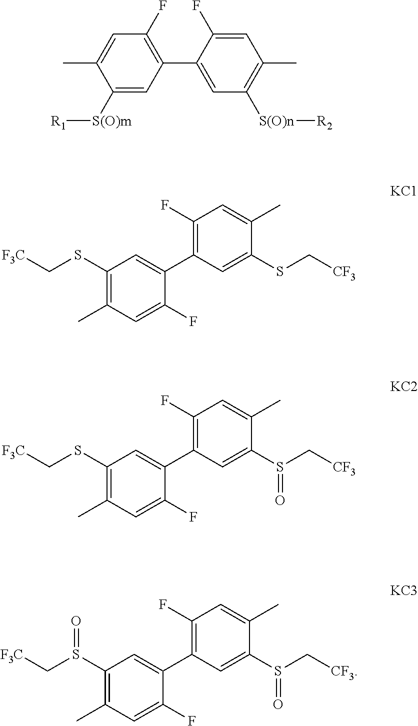

In the prior art, a compound shown in Formula I of the present invention and insecticidal and acaricidal activities thereof have not been reported. Moreover, compared with the prior art, the compound of the present invention has higher insecticidal and acaricidal activities.

### SUMMARY OF THE INVENTION

An objective of the present invention is to provide a biphenyl compound containing substituted sulfide (sulfoxide) groups having better insecticidal and acaricidal effects, which can be used for preventing and treating pests and pest mites in the fields of agriculture, forestry, and health. The technical solutions of the present invention are described below.

A biphenyl compound containing substituted sulfide (sulfoxide) groups is shown in Formula I:

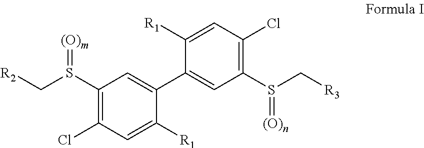

wherein:
R₁ is selected from hydrogen or a halogen;
R₂ and R₃ are each independently selected from hydrogen, cyano, cyclopropyl, C₁-C₃ alkyl, or C₁-C₃ haloalkyl;
m and n are each independently selected from 0 or 1.

In a possible embodiment, in Formula I,
R₁ is selected from hydrogen, fluorine, chlorine, or bromine;
R₂ and R₃ are each independently selected from hydrogen, cyano, cyclopropyl, methyl, ethyl, n-propyl, isopropyl, —CF₃, —CHF₂, —CH₂F, —CH₂CF₃, —CH₂CHF₂, or —CH₂CH₂F;
m and n are each independently selected from 0 or 1.

In a possible embodiment, in Formula I,
R₁ is selected from fluorine;
R₂ and R₃ are each independently selected from hydrogen, cyano, cyclopropyl, methyl, ethyl, n-propyl, isopropyl, —CF₃, —CHF₂, —CH₂F, —CH₂CF₃, —CH₂CHF₂, or —CH₂CH₂F;
m and n are each independently selected from 0 or 1.

In a possible embodiment, in Formula I,
R₁ is selected from fluorine;
R₂ and R₃ are selected from —CF₃;
m and n are each independently selected from 0 or 1.

An intermediate compound for preparing the biphenyl compound containing substituted sulfide (sulfoxide) groups described above is shown in Formula II:

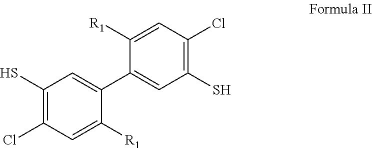

wherein:
R₁ is selected from hydrogen or a halogen.

A intermediate compound for preparing the biphenyl compound containing substituted sulfide (sulfoxide) groups described above is shown in Formula III:

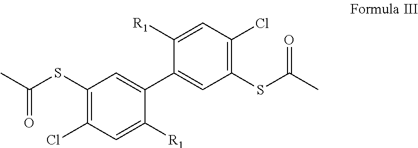

wherein:
R₁ is selected from hydrogen or a halogen.

A intermediate compound for preparing the compound of Formula II or the compound of Formula III described above are shown in Formula IV:

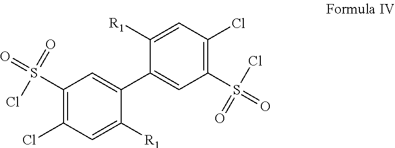

wherein:
R₁ is selected from hydrogen or a halogen.

In the definitions of the compound of Formula I, the compound of Formula II, the compound of Formula III, and the compound of Formula IV described above, the halogen refers to fluorine, chlorine, bromine, or iodine; the alkyl refers to a linear or branched alkyl, such as methyl, ethyl, n-propyl, isopropyl, or different butyl isomers; the haloalkyl refers to a linear or branched alkyl, and hydrogen atoms on such an alkyl may be partly or totally substituted by a halogen, such as —CF₃, —CHF₂, —CH₂F, —CH₂CF₃, —CH₂CHF₂, or —CH₂CH₂F.

Partial compounds of Formula I of the present invention are shown in Table 1 to Table 10, but the present invention is by no means limited to these compounds.

### Table 1

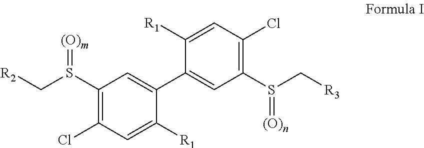

In Formula I, when R₁=F and R₂=R₃, R₂ or R₃ is a different substituent, and m and n are different numbers, as shown in Table 1. Representative compounds are numbered from 1.1 to 1.39.

| No. | R₂ or R₃ | m | n | No. | R₂ or R₃ | m | n | No. | R₂ or R₃ | m | n |
|---|---|---|---|---|---|---|---|---|---|---|---|
| 1.1 | H | 0 | 0 | 1.2 | H | 0 | 1 | 1.3 | H | 1 | 1 |
| 1.4 | Cyano | 0 | 0 | 1.5 | Cyano | 0 | 1 | 1.6 | Cyano | 1 | 1 |
| 1.7 | Cyclopropyl | 0 | 0 | 1.8 | Cyclopropyl | 0 | 1 | 1.9 | Cyclopropyl | 1 | 1 |
| 1.10 | Methyl | 0 | 0 | 1.11 | Methyl | 0 | 1 | 1.12 | Methyl | 1 | 1 |
| 1.13 | Ethyl | 0 | 0 | 1.14 | Ethyl | 0 | 1 | 1.15 | Ethyl | 1 | 1 |
| 1.16 | N-propyl | 0 | 0 | 1.17 | N-propyl | 0 | 1 | 1.18 | N-propyl | 1 | 1 |
| 1.19 | Isopropyl | 0 | 0 | 1.20 | Isopropyl | 0 | 1 | 1.21 | Isopropyl | 1 | 1 |
| 1.22 | —CF₃ | 0 | 0 | 1.23 | —CF₃ | 0 | 1 | 1.24 | —CF₃ | 1 | 1 |
| 1.25 | —CHF₂ | 0 | 0 | 1.26 | —CHF₂ | 0 | 1 | 1.27 | —CHF₂ | 1 | 1 |
| 1.28 | —CH₂F | 0 | 0 | 1.29 | —CH₂F | 0 | 1 | 1.30 | —CH₂F | 1 | 1 |
| 1.31 | —CH₂CF₃ | 0 | 0 | 1.32 | —CH₂CF₃ | 0 | 1 | 1.33 | —CH₂CF₃ | 1 | 1 |
| 1.34 | —CH₂CHF₂ | 0 | 0 | 1.35 | —CH₂CHF₂ | 0 | 1 | 1.36 | —CH₂CHF₂ | 1 | 1 |
| 1.37 | —CH₂CH₂F | 0 | 0 | 1.38 | —CH₂CH₂F | 0 | 1 | 1.39 | —CH₂CH₂F | 1 | 1 |

Table 2: in Formula I, when R₁=H and R₂=R₃, R₂ or R₃ is a different substituent, and m and n are different numbers, which are consistent with those in Table 1. Representative compounds are numbered from 2.1 to 2.39, corresponding to 1.1 to 1.39 in Table 1 in sequence.

Table 3: in Formula I, when R₁=Cl and R₂=R₃, R₂ or R₃ is a different substituent, and m and n are different numbers, which are consistent with those in Table 1. Representative compounds are numbered from 3.1 to 3.39, corresponding to 1.1 to 1.39 in Table 1 in sequence.

Table 4: in Formula I, when R₁=Br and R₂=R₃, R₂ or R₃ is a different substituent, and m and n are different numbers, which are consistent with those in Table 1. Representative compounds are numbered from 4.1 to 4.39, corresponding to 1.1 to 1.39 in Table 1 in sequence.

Table 5: in Formula I, when R₁=I and R₂=R₃, R₂ or R₃ is a different substituent, and m and n are different numbers, which are consistent with those in Table 1. Representative compounds are numbered from 5.1 to 5.39, corresponding to 1.1 to 1.39 in Table 1 in sequence.

In Formula I, when R₁=F and R₂=—CF₃, R₃ is a different substituent, and m and n are different numbers, as shown in Table 6. Representative compounds are numbered from 6.1 to 6.36.

TABLE 6

| No. | R₃ | m | n | No. | R₃ | m | n | No. | R₃ | m | n |
|---|---|---|---|---|---|---|---|---|---|---|---|
| 6.1 | H | 0 | 0 | 6.2 | H | 0 | 1 | 6.3 | H | 1 | 1 |
| 6.4 | Cyano | 0 | 0 | 6.5 | Cyano | 0 | 1 | 6.6 | Cyano | 1 | 1 |
| 6.7 | Cyclopropyl | 0 | 0 | 6.8 | Cyclopropyl | 0 | 1 | 6.9 | Cyclopropyl | 1 | 1 |
| 6.10 | Methyl | 0 | 0 | 6.11 | Methyl | 0 | 1 | 6.12 | Methyl | 1 | 1 |
| 6.13 | Ethyl | 0 | 0 | 6.14 | Ethyl | 0 | 1 | 6.15 | Ethyl | 1 | 1 |
| 6.16 | N-propyl | 0 | 0 | 6.17 | N-propyl | 0 | 1 | 6.18 | N-propyl | 1 | 1 |
| 6.19 | Isopropyl | 0 | 0 | 6.20 | Isopropyl | 0 | 1 | 6.21 | Isopropyl | 1 | 1 |
| 6.22 | —CHF₂ | 0 | 0 | 6.23 | —CHF₂ | 0 | 1 | 6.24 | —CHF₂ | 1 | 1 |
| 6.25 | —CH₂F | 0 | 0 | 6.26 | —CH₂F | 0 | 1 | 6.27 | —CH₂F | 1 | 1 |
| 6.28 | —CH₂CF₃ | 0 | 0 | 6.29 | —CH₂CF₃ | 0 | 1 | 6.30 | —CH₂CF₃ | 1 | 1 |
| 6.31 | —CH₂CHF₂ | 0 | 0 | 6.32 | —CH₂CHF₂ | 0 | 1 | 6.33 | —CH₂CHF₂ | 1 | 1 |
| 6.34 | —CH₂CH₂F | 0 | 0 | 6.35 | —CH₂CH₂F | 0 | 1 | 6.36 | —CH₂CH₂F | 1 | 1 |

Table 7: in Formula I, when R₁=H and R₂=—CF₃, R₃ is a different substituent, and m and n are different numbers, which are consistent with those in Table 1. Representative compounds are numbered from 7.1 to 7.36, corresponding to 6.1 to 6.36 in Table 6 in sequence.

Table 8: in Formula I, when R₁=Cl and R₂=—CF₃, R₃ is a different substituent, and m and n are different numbers, which are consistent with those in Table 1. Representative compounds are numbered from 8.1 to 8.36, corresponding to 6.1 to 6.36 in Table 6 in sequence.

Table 9: in Formula I, when R₁=Br and R₂=—CF₃, R₃ is a different substituent, and m and n are different numbers, which are consistent with those in Table 1. Representative compounds are numbered from 9.1 to 9.36, corresponding to 6.1 to 6.36 in Table 6 in sequence.

Table 10: in Formula I, when R₁=I and R₂=—CF₃, R₃ is a different substituent, and m and n are different numbers, which are consistent with those in Table 1. Representative compounds are numbered from 10.1 to 10.36, corresponding to 6.1 to 6.36 in Table 6 in sequence.

Partial compounds of Formula II of the present invention are shown in Table 11, but the present invention is by no means limited to these compounds.

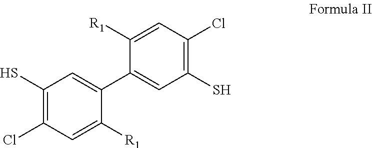

TABLE 11

| No. | R₁ | No. | R₁ | No. | R₁ | No. | R₁ | No. | R₁ |
|---|---|---|---|---|---|---|---|---|---|
| 11.1 | H | 11.2 | F | 11.3 | Cl | 11.4 | Br | 11.5 | I |

Partial compounds of Formula III of the present invention are shown in Table 12, but the present invention is by no means limited to these compounds.

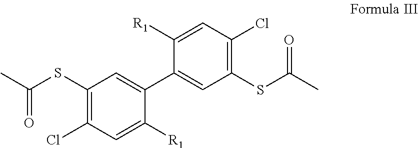

TABLE 12

| No. | R₁ | No. | R₁ | No. | R₁ | No. | R₁ | No. | R₁ |
|---|---|---|---|---|---|---|---|---|---|
| 12.1 | H | 12.2 | F | 12.3 | Cl | 12.4 | Br | 12.5 | I |

Partial compounds of Formula IV of the present invention are shown in Table 13, but the present invention is by no means limited to these compounds.

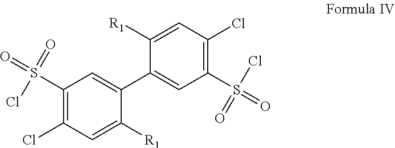

TABLE 13

| No. | R₁ | No. | R₁ | No. | R₁ | No. | R₁ | No. | R₁ |
|---|---|---|---|---|---|---|---|---|---|
| 13.1 | H | 13.2 | F | 13.3 | Cl | 13.4 | Br | 13.5 | I |

The compound of Formula I, the compound of Formula II, the compound of Formula III, and the compound of Formula IV of the present invention may be prepared according to the following schemes, and the definitions of each group in the formulas are the same as before unless otherwise indicated.

Scheme I is intended to prepare the compound of Formula I, the compound of Formula II, the compound of Formula III, and the compound of Formula IV with m=n=0.

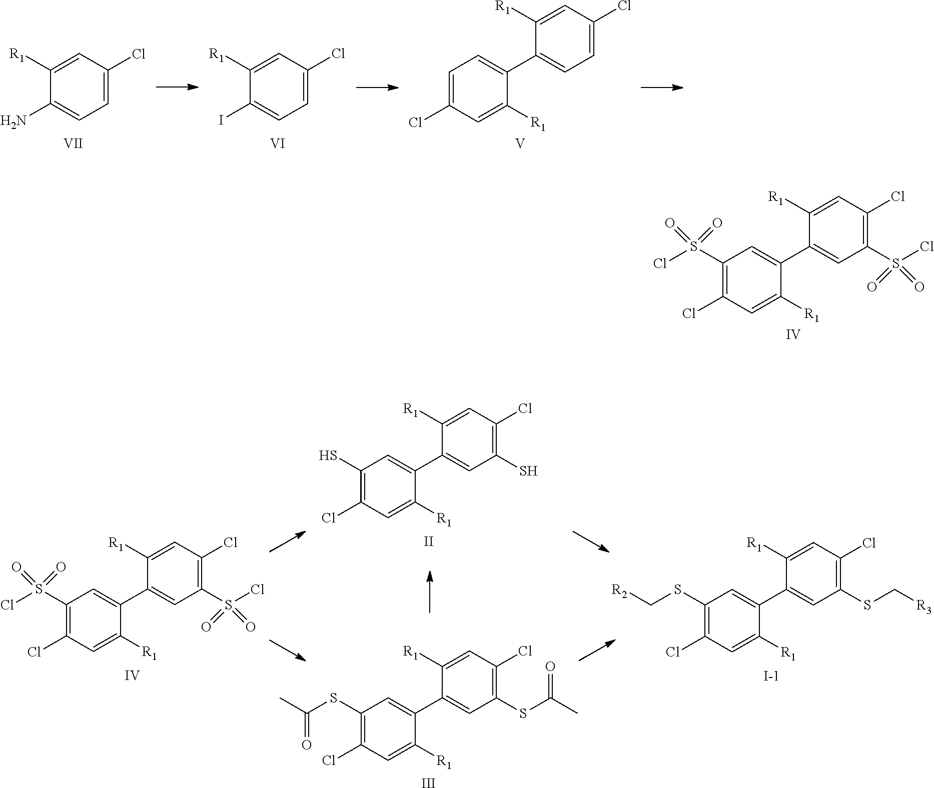

A compound of Formula VI may be prepared through a reaction of the compound of Formula VII with sodium nitrite, one or more acids, and potassium iodide. The acids may be inorganic acids or organic acids, such as hydrochloric acid, hydrobromic acid, sulfuric acid, phosphoric acid, formic acid, acetic acid, propionic acid, butyric acid, valeric acid, trifluoroacetic acid, oxalic acid, malonic acid, or methanesulfonic acid, or the like. A reactive solvent may be water, chloroform, dichloromethane, carbon tetrachloride, hexane, benzene, toluene, ethyl acetate, DMF, tetrahydrofuran, dioxane, or the like. The reaction usually takes place at a temperature of 0° C. to 100° C. The reaction usually lasts for 0.5 to 48 hours.

The compound of Formula VII can be commercially available.

A compound of Formula V may be prepared through a reaction of the compound of Formula VI with bis(pinacolato)diboron in a suitable solvent in the presence of a suitable base and a suitable palladium catalyst at a temperature from −10° C. to a boiling point of the solvent for 0.5 to 48 hours. The suitable solvent may be selected from water, dichloromethane, chloroform, carbon tetrachloride, hexane, benzene, toluene, acetonitrile, tetrahydrofuran, 1,4-dioxane, DMF, DMSO, or the like. The suitable base includes a hydride of an alkali metal such as lithium, sodium, or potassium, such as sodium hydride or potassium hydride, and a hydroxide of an alkali metal such as lithium, sodium, or potassium, such as sodium hydroxide or potassium hydroxide, may be a carbonate of an alkali metal such as lithium, sodium, potassium, or cesium, such as sodium carbonate or cesium carbonate, and may also be an organic base such as triethylamine, sodium tert-butoxide, or potassium tert-butoxide. The suitable palladium catalyst may be tetrakis (triphenylphosphine) palladium (0), [1,l′-bis(diphenylphosphino) ferrocene]dichloropalladium (II), or the like. In some cases, a suitable ligand such as 1,1′-bis(diphenylphosphino) ferrocene, triphenylphosphine, and tri-tert-butylphosphine may also be added.

A compound of Formula IV may be prepared through a reaction of the compound of Formula V with a sulfonating reagent. The reaction usually takes place at a temperature of 0° C. to 200° C. (such as 190° C., 170° C., 150° C., 130° C., 110° C., 100° C., 90° C., 80° C., 70° C., 60° C., 50° C., and 30° C.). The reaction usually lasts for 0.5 to 48 hours. The sulfonating reagent may be chlorosulfonic acid, fuming sulfuric acid, concentrated sulfuric acid, sulfur trioxide, sulfur monochloride, or the like. A feeding molar ratio of the compound of Formula V to the sulfonating reagent is 1:1-100 (such as 1:1-90, 1:1-80, 1:1-70, 1:1-60, 1:1-50, 1:1-40, 1:1-30, 1:1-20, 1:1-10, 1:1-8, 1:1-6, 1:1-4, 1:1-3, and 1:1-2).

The compound of Formula II or the compound of Formula III may be prepared through a reaction of the compound of Formula IV with a reducing reagent. The reaction usually takes place at a temperature of 0° C. to 150° C. (such as 140° C., 130° C., 120° C., 110° C., 100° C., 90° C., 80° C., 70° C., 60° C., 50° C., and 30° C.). The reaction usually lasts for 0.5 to 48 hours. The reducing reagent may be red phosphorus, zinc, iron, copper, or nickel, or a mixture of red phosphorus, zinc, iron, copper, and nickel in any ratio. A feeding molar ratio of the compound of Formula IV to the reducing reagent is 1:1-30 (such as 1:1-25, 1:1-20, 1:1-10, 1:1-9, 1:1-8, 1:1-7, 1:1-6, 1:1-5, 1:1-4, 1:1-3, and 1:1-2). A suitable amount of an organic acid or an inorganic acid such as formic acid, acetic acid, trifluoroacetic acid, hydrochloric acid, sulfuric acid or nitric acid may be added in the reaction. A feeding molar ratio of the compound of Formula IV to the acid is 1:1-100 (such as 1:1-90, 1:1-80, 1:1-70, 1:1-60, 1:1-50, 1:1-40, 1:1-30, 1:1-20, 1:1-10, 1:1-9, 1:1-8, 1:1-7, 1:1-6, 1:1-5, 1:1-4, 1:1-3, and 1:1-2).

The compound of Formula II may be prepared by subjecting the compound of Formula III to conventional acidic hydrolysis or basic hydrolysis.

A compound of Formula I-1 may be prepared through a reaction of the compound of Formula II or the compound of Formula III with a halogenating reagent or a sulfonate. The halogenating reagent may be trifluoroiodoethane, iodomethane, iodoethane, or the like. The sulfonate may be 2,2,2-trifluoroethyl methanesulfonate, 2,2,2-trifluoroethyl benzenesulfonate, 2,2,2-trifluoroethyl p-toluenesulfonate, or the like. A feeding molar ratio of the compound of Formula II or the compound of Formula III to the halogenating reagent is 1:1-100 (such as 1:1-90, 1:1-80, 1:1-70, 1:1-60, 1:1-50, 1:1-40, 1:1-30, 1:1-20, 1:1-10, 1:1-9, 1:1-8, 1:1-7, 1:1-6, 1:1-5, 1:1-4, 1:1-3, and 1:1-2). The suitable base may be a same or different organic base such as trimethylamine, triethylamine, pyridine, DBU, 4-dimethylaminopyridine, N,N-diisopropylethylamine, or the like, alkali metal hydride such as sodium hydride and potassium hydride, alkali metal hydroxide such as sodium hydroxide and potassium hydroxide, alkaline-earth metal hydroxide such as calcium hydroxide, alkali metal carbonate such as sodium carbonate and potassium carbonate, alkali metal bicarbonate such as sodium bicarbonate, or metal alkoxide such as sodium methoxide, sodium ethoxide, potassium ethoxide, potassium tert-butoxide, and sodium tert-butoxide. A feeding molar ratio of the compound of Formula II or the compound of Formula III to the base is 1:1-20 (such as 1:1-18, 1:1-16, 1:1-14, 1:1-12, 1:1-10, 1:1-9, 1:1-8, 1:1-7, 1:1-6, 1:1-5, 1:1-4, 1:1-3, and 1:1-2). The suitable solvent may be a same or different aromatic hydrocarbon such as benzene, toluene, and xylene, ketone such as acetone, methyl ethyl ketone, and methyl isobutyl ketone, halohydrocarbon such as chloroform and dichloromethane, ester such as methyl acetate and ethyl acetate, ether such as tetrahydrofuran, dioxane, diethyl ether, 1,2-dimethoxyethane, and 1,4-dioxane, or polar solvent such as water, acetonitrile, N,N-dimethylformamide, N-methylpyrrolidone, and dimethyl sulfoxide, or a mixed solvent of such solvents. The reaction usually takes place at 0° C. to a boiling point of the solvent. The reaction usually lasts for 0.5 to 48 hours.

Scheme II is intended to prepare the compound of Formula I with m=1 or n=1, and m=n=1.

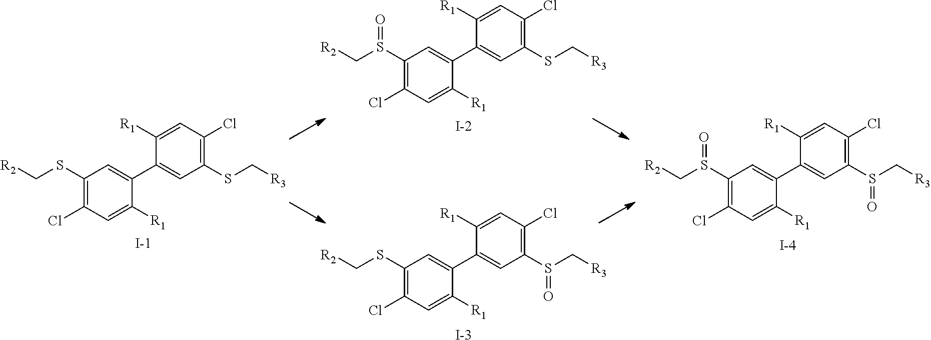

A compound of Formula I-2 may be prepared through a reaction of the compound of Formula I-1 with an oxidizing agent, and a compound of Formula I-4 may be prepared through a further reaction of the compound of Formula I-2 with an oxidizing agent. Likewise, a compound of Formula I-3 may be prepared through a reaction of the compound of Formula I-1 with an oxidizing agent, and the compound of Formula I-4 may be prepared through a further reaction of the compound of Formula I-3 with an oxidizing agent. The oxidizing agent may be metachloroperbenzoic acid, hydrogen peroxide, sodium (meta) periodate, or the like. A reactive solvent may be water, methanol, ethanol, ethyl ether, dichloromethane, chloroform, carbon tetrachloride, hexane, benzene, toluene, ethyl acetate, DMF, tetrahydrofuran, dioxane, or the like. The reaction usually takes place at a temperature of 0° C. to 100° C., preferably 0° C. to 30° C. The reaction usually lasts for 10 minutes to 48 hours.

The compound of Formula I of the present invention exhibits an effective insecticidal effect. Therefore, the compound of Formula I of the present invention can be used as insecticides. The compound of Formula I of the present invention also exhibits an appropriate prevention and treatment effect for poisonous pests, and has no phytotoxicity for plants such as cultivated crops. Moreover, the compound of the present invention may be used for preventing and treating a variety of pests, such as harmful piercing-sucking insects, chewing insects, and other plant parasitic pests, storage cereal pests, sanitary pests, etc., and can be used for sterilizing and killing them.

Such harmful insects may be described below by way of example.

As insects,

Beetles (Coleopteran), such as Callosobruchus chinensis, Sitophilus zeamais, Tribolium castaneum, Epilachna vigintioctomaculata, Agriotes ogurae fuscicollis, Anomala rufocuprea, Leptinotarsa decemlineata, Diabrotica spp., Monochamus alternatus endai, Lissorhoptrus oryzophilus, and Lyctus bruneus

Lepidopteran pests, such as Lymantria dispar, Malacosoma neustria, Pieris rapae crucivora, Spodoptera litura, Mamestra brassicae, Chilo suppressalis, Ostrinia nubilalis, Cadra cautella, Chyanokokakumonhamaki (Adoxophyes honmai), Cydia pomonella, Agrotis segetum, Galleria mellonella, Plutella xylostella, Heliothis virescens, and Phyllocnistis citrella;

Hemipterous pests, such as Nephotettix cincticeps, Nilaparvata lugens, Pseudococcus comstocki, Unaspis yanonensis, Myzus persicas, Aphis pomi, Aphis gossypii, Lipaphis erysimi, Stephanitis nashi, Nezara spp., Trialeurodes vaporariorum, and Pshylla spp.;

Thysanoptera pests, such as Thrips palmi and Franklinella occidentalis;

Orthopteran pests, such as Gryllotalpa africana and Locusta migratoria;

Blattarian pests, such as Blattella germanica, Periplaneta americana, Reticulitermes speratus, and Coptotermes formosanus;

Dipterous pests, such as Musca domestica, Aedesaegypti, Delia platura, Culex pipiens pallens, Anopheles sinensis, Culex tritaeniorhynchus, and Liriomyza trifolii; or the like.

Moreover, the present invention further includes a use of the compound of Formula I for controlling pest mites. In particular, the compound of Formula I has activity for important species of the following families: Tetranychidae (Tetranychus urticae koch, Tetranychus cinnabarinus, Panonychus Ulmi, Panonychus citri, or the like), Eriophyidae, Tarsonemidae, or the like.

Moreover, as nematodes, Meloidogyne incognita, Bursaphelenchus xylophilus, Aphelenchoides besseyi, Heterodera glycines, Pratylenchus spp., or the like, may be referred.

In the present invention, substances having an insecticidal effect for harmful pests including all such pests are referred to as insecticides.

When used as insecticides, the active compound of the present invention may be prepared in the form of common formulations. The forms of formulations may include, for example, solutions, emulsions, wettable powders, granular wettable powders, suspensions, powders, foams, plasters, tablets, granules, aerosols, natural reagents impregnated with active compounds, synthetic reagents impregnated with active compounds, microcapsules, seed coating agents, formulations equipped with combustion devices (the combustion devices may be a smoke tube, a spray tube, a canister, a coil, or the like), and ULV (cold sprays and hot sprays).

Such formulations may be prepared by well-known methods. For example, they may be prepared by mixing the active compound with a filler (i.e., a liquid diluter or carrier, a liquefied gas diluter or carrier, a solid diluter or carrier), and optionally with a surfactant (i.e., an emulsifier and/or a dispersant and/or a foaming agent) or the like.

When water is used as the filler, for example, an organic solvent may be used as a cosolvent. The liquid diluter or carrier may include, for example, aromatic hydrocarbons (xylene, toluene, alkyl naphthalene, etc.), chlorinated aromatic hydrocarbons or chlorinated aliphatic hydrocarbons (such as chlorobenzene, chloroethylene, and dichloromethane), aliphatic hydrocarbons (such as cyclohexane or paraffin (e.g., mineral oil distillate)), alcohols (such as butanol, glycol, and ethers or esters thereof), ketones (such as acetone, methyl ethyl ketone, methyl isobutyl ketone, and cyclohexanone), strong polar solvents (such as dimethylformamide and dimethylsulfoxide), and water.

The liquefied gas diluter or carrier may include those existing in a gaseous form under atmospheric pressure and at atmospheric temperature, such as propane, nitrogen, carbon dioxide, and aerosol propellants such as halohydrocarbons.

Examples of the solid diluter may include pulverized natural minerals (such as kaolin, clay, talcum, greda, quartz, attapulgite, montmorillonite, or diatomite), pulverized synthetic minerals (such as fine dispersed silicic acid, aluminum oxide, and silicate), or the like.

Examples of the solid diluter for granules may include pulverized and graded rocks (such as calcite, marble, pumice stone, sepiolite, and dolomite), particles of synthetic organic or organic powders, fine particles of organic materials (such as saw dust, coconut shell, corncob, and tobacco stalk), or the like.

Examples of the emulsifier and/or the foaming agent may include nonionic and anionic emulsifiers (such as polyoxyethylene fatty acid esters, polyoxyethylene fatty acid alcohol ethers (e.g., alkylarylpolyglycol ether), alkane sulfonates, alkyl sulfates, and arylsulphonates), albumin hydrolysates, or the like.

The dispersant includes lignin sulfite liquid waste and methylcellulose.

An adhesive may also be used in the formulations (powders, granules, and emulsions). Examples of the adhesive may include carboxymethylcellulose, and natural or synthetic polymers (such as acacia gum, polyvinyl alcohol, and polyvinyl acetate).

A colorant may also be used. Examples of the colorant may include inorganic pigments (such as iron oxide, titanium oxide, and Prussian blue), organic dyes such as alizarin dye, azo dye, or metal phthalocyanine dye; and microelements such as iron salts, manganese salts, boron salts, copper salts, cobalt salts, molybdenum salts, or zinc salts.

The formulations may contain the above-mentioned active component in an amount of 0.1% to 99% by weight, preferably 0.5% to 90% by weight.

The compound of Formula I of the present invention may be used as a mixture with other active compounds (such as an insecticide, a poison bait, a disinfectant, an acaricide, a nematicide, a fungicide, a growth regulator, and a herbicide) and provided in a form of commercially available useful formulations or in a use form prepared from formulations thereof. The insecticide may include, for example, an organophosphorus reagent, a carbaminate reagent, a carboxylate reagent, a chlorinated hydrocarbon reagent, and an insecticidal substance prepared from microorganisms. Moreover, the compound of Formula I of the present invention may be present in a form of a mixture with a synergist. The formulations and use forms may include those commercially available. The synergist needs not to be active itself. To be more exact, it is a compound that enhances the activity of the active compound.

The amount of the included compound of Formula I of the present invention in the commercially available form may vary within a wide range.

An actual use concentration of the compound of Formula I of the present invention may be, for example, between 0.0000001% and 100% by weight, preferably between 0.00001% and 1% by weight.

The compound of Formula I of the present invention may be used by any conventional method suitable for the use form.

When the active compound of the present invention is used for resisting sanitary pests and storage pests, the compound has an effective stability for alkaline substances existing in liming materials. Moreover, the compound of the present invention exhibits an excellent residual efficiency in wood and soil.

The active compound of the present invention has hypotoxicity and can be safely used for warm-blooded animals.

It should be specified that various alterations and modifications may be made within the scope defined by the claims of the present invention.

### DETAILED DESCRIPTION OF THE INVENTION

The following specific examples are provided to further describe the present invention, but the present invention is by no means limited to such examples. (Unless otherwise indicated, all raw materials used are commercially available.)

### SYNTHESIS EXAMPLES

According to the synthesis schemes described above, the compounds of Formula I, Formula II, Formula III, and Formula IV of the present invention may be prepared by using different raw material compounds, respectively, which will be further described specifically below.

### Example 1: The Preparation of Compound 1.22

### (1) Preparation of Intermediate 4-chloro-2-fluoro-1-iodobenzene (VI-1)

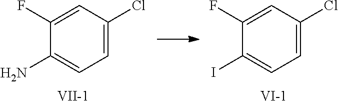

5.00 g (34.35 mmol) of 4-chloro-2-fluoroaniline (VII-1) was weighted and added into 200 mL of water and 35 mL of concentrated hydrochloric acid, cooled to a range of 0° C. to 5° C., and stirred for 30 min. 100 mL of an aqueous solution of 2.40 g (34.79 mmol) of sodium nitrite was added dropwise thereto, during which the reaction temperature was maintained not to exceed a range of 0° C. to 5° C. After the completion of dropwise addition, stirring was continued for reaction for 1 h.

100 mL of an aqueous solution of 5.70 g (34.34 mmol) of potassium iodide was added dropwise into the diazonium salt solution, during which the reaction temperature was maintained not to exceed a range of 0° C. to 5° C. After the completion of dropwise addition, an ice bath was removed and stirring was continued at room temperature for reaction for 3 h. After the reaction was finished under TLC monitoring, 300 mL of ethyl acetate was added thereto for dilution. The organic layer was washed with 200 ml of water and 200 mL of saturated salt solution in sequence, dried with anhydrous magnesium sulfate, filtered, and desolventized under reduced pressure, and the residue was purified by column chromatography to obtain 5.70 g of a rufous liquid, i.e., the intermediate VI-1.

### (2) Preparation of Intermediate 4,4′-dichloro-2,2′-difluoro-1,1′-biphenyl (V-1)

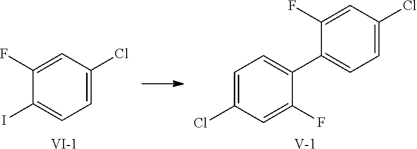

5.00 g (19.54 mmol) of the intermediate 4-chloro-2-fluoro-1-iodobenzene (VI-1), 7.44 g (29.31 mmol) of bis(pinacolato)diboron, 12.74 g (39.08 mmol) of cesium carbonate, 0.09 g (0.12 mmol) of [1,1′-bis(diphenylphosphino) ferrocene]dichloropalladium (II), and 0.06 g (0.10 mmol) of 1,1′-bis(diphenylphosphino) ferrocene were added into 100 mL of 1,4-dioxane and 3 mL of water, and subjected to heating reflux for reaction for 3 h. After the reaction was finished under TLC monitoring, 200 mL of ethyl acetate was added thereto for dilution. The organic layer was washed with 200 mL of water and 200 mL of saturated salt solution in sequence, dried with anhydrous magnesium sulfate, filtered, and desolventized under reduced pressure. The residue was purified by column chromatography to obtain 2.00 g of a white solid, i.e., the intermediate V-1.

### (3) Preparation of Intermediate 4,4′-dichloro-6,6′-difluoro-[1,1′-biphenyl]-3,3′-disulfonyl dichloride (IV-1)

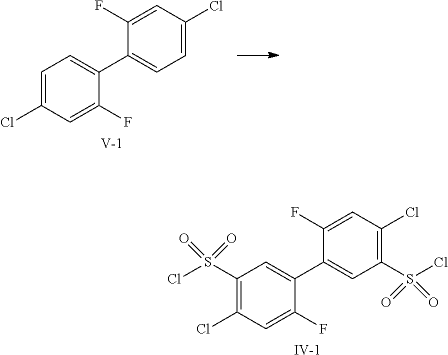

481.6 g (4.14 mol) of chlorosulfonic acid was added to a reaction flask, cooled using ice water to a range of 0° C. to 5° C., and 50 g (0.19 mol) of the intermediate 4,4′-dichloro-2,2′-difluoro-1,1′-biphenyl (V-1) was added thereto, during which the temperature of the reaction mixture did not exceed 10° C. The temperature was increased to a range of 25° C. to 30° C. and maintained for reaction for 3 h. After the reaction was finished under TLC monitoring, the reaction mixture was slowly poured into crushed ice, and a solid was precipitated, which was filtered, and the filtrate was extracted with ethyl acetate and water. The organic layer was washed with saturated salt solution, dried with anhydrous magnesium sulfate, filtered, and desolventized under reduced pressure to obtain a yellowy solid. The yellowy solid was combined with the filter cake, and dried to obtain 56.0 g of another yellowy solid, i.e., the intermediate IV-1. Nuclear magnetic resonance (NMR) data of the intermediate III-1 were shown below:

¹H NMR (600 MHz, DMSO-d₆) δ 7.92-7.86 (m, 2H), 7.55-7.50 (m, 2H).

### (4) Preparation of Intermediate 4,4′-dichloro-6,6′-difluoro-[1,1′-biphenyl]-3,3′-dithiol (II-1)

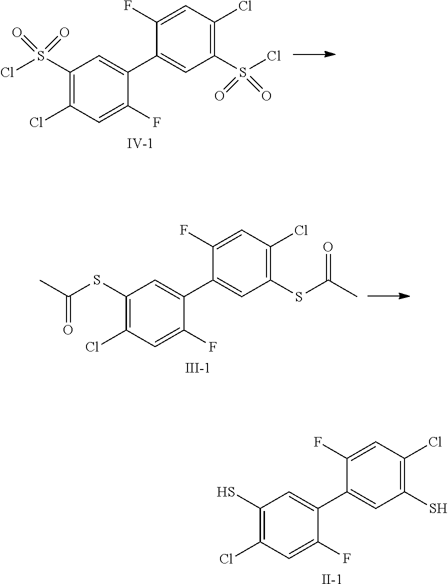

0.70 g (11.69 mmol) of acetic acid, 0.39 g (0.86 mmol) of the intermediate 4,4′-dichloro-6,6′-difluoro-[1,1′-biphenyl]-3,3′-disulfonyl dichloride (IV-1), 0.16 g (5.16 mmol) of red phosphorus, 0.02 g (0.09 mmol) of iodine, and 0.35 g (3.44 mmol) of acetic anhydride were added into a reaction flask in sequence, and warmed for reflux reaction for 1 h. After the reaction was finished under TLC monitoring, the reaction mixture was hot filtered, and the mother solution was concentrated, and extracted with 100 mL of ethyl acetate and 100 mL of water such that the mother solution was layered. The organic layer was added with sodium bicarbonate to adjust the pH to a range of 6 to 7. The organic layer was then concentrated under reduced pressure to obtain 0.28 g of a yellow solid, i.e., the intermediate III-1.

0.28 g of the yellow solid (III-1), 10 mL of tetrahydrofuran, and 0.12 g (12.86 mmol) of sodium formaldehyde sulfoxylate were added to a reaction flask, and cooled using an ice-water bath to a range of 0° C. to 5° C. An aqueous solution of sodium hydroxide (0.17 g dissolved in 10 mL of water) was added dropwise thereto, during which the temperature was controlled to a range of 0° C. to 5° C. After the completion of dropwise addition, stirring was continued for reaction for 30 min. After the reaction was finished under TLC monitoring, 20 mL of water and 20 mL of ethyl acetate were added into the reaction mixture for extraction and layering, and the organic phase was removed. Concentrated hydrochloric acid (0.44 g, 4.30 mmoL) was added dropwise into the water phase. After the completion of dropwise addition, stirring was continued for 30 min, during which a solid was precipitated continuously. 20 mL of ethyl acetate was added thereto for extraction, and the organic phase was dried with anhydrous magnesium sulfate and concentrated under reduced pressure to obtain 0.17 g of a white solid, i.e., the intermediate II-1.

### (5) Preparation of Compound 1.22

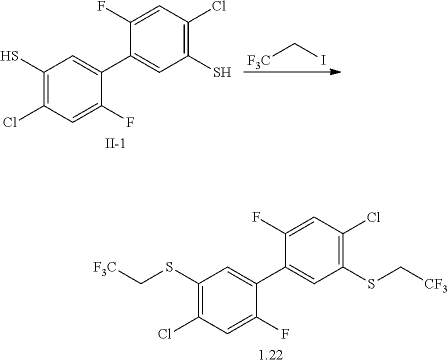

8.00 g (24.85 mmol) of the intermediate 4,4′-dichloro-6,6′-difluoro-[1,1′-biphenyl]-3,3′-dithiol (II-1), 80 mL of acetone, 11.48 g (54.67 mmol) of potassium carbonate, 3.23 g (27.34 mmol) of sodium formaldehyde hydrosulfite, and 11.48 g (54.67 mmol) of trifluoroiodoethane were added into a reaction flask in sequence, and the reaction mixture was warmed to 50° C. for reaction for 5 h. After the reaction was finished under TLC monitoring, the reaction mixture was concentrated, and the residue was purified by column chromatography to obtain 8.45 g of a white solid, i.e., the target compound 1.22. The NMR data of the compound 1.22 were shown below:

¹H NMR (400 MHz, Chloroform-d) δ 7.65-7.61 (m, 2H), 7.36-7.32 (m, 2H), 3.45 (q, 4H).

### Example 2: Preparation of Compounds 1.23 and 1.24

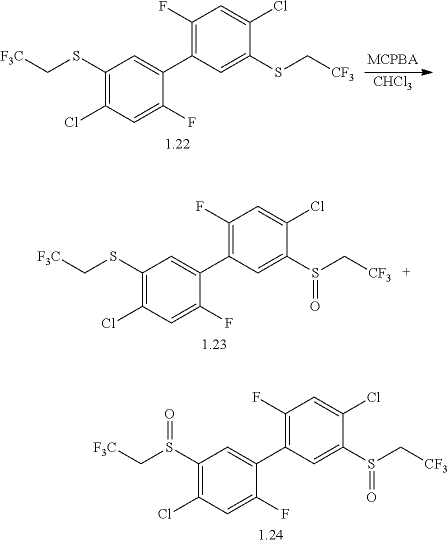

0.30 g (0.62 mmol) of the compound 1.22 was dissolved in 10 mL of chloroform, cooled to a range of 0° C. to 5° C., then 0.12 g (0.70 mmol, with a purity of 85%) of metachloroperbenzoic acid was added thereto, and stirred for reaction for 1 h. After the reaction was finished under TLC monitoring, the reaction mixture was washed with an aqueous solution of sodium thiosulfate and an aqueous solution of sodium bicarbonate in sequence, dried with anhydrous magnesium sulfate, filtered, and desolventized under reduced pressure. The residue was purified by column chromatography to obtain 0.16 g of the compound 1.23 (a white solid) and 0.12 g of the compound 1.24 (a white solid). The NMR data were shown below:

Compound 1.23: ¹H NMR (600 MHz, Chloroform-d) δ 7.98 (d, 1H), 7.68 (d, 1H), 7.40-7.34 (m, 2H), 3.84-3.74 (m, 1H), 3.51-3.40 (m, 3H).

Compound 1.24: ¹H NMR (600 MHz, Chloroform-d) δ 8.05-8.00 (m, 2H), 7.41-7.36 (m, 2H), 3.84-3.73 (m, 2H), 3.53-3.41 (m, 2H).

Other compounds of Formula I of the present invention may be prepared with reference to the above examples.

### Bioactivity Determination

### Example 3: Bioactivity Determination on Mythimna separata and Plutella xylostella

Insecticidal activity determination experiments were conducted on several insects using the compound of the present invention and control compounds KC1, KC2, and KC3. Determination methods were described below.

The compounds to be determined were each dissolved in a mixed solvent of acetone/methanol (1:1), and diluted using water containing 0.1% (wt) tween-80 to desired concentrations.

With Mythimna separata and Plutella xylostella as targets, Airbrush spray method was adopted for activity determination.

### (1) Determination of Activity Against Mythimna Separata

Determination Method: maize leaves were cut into 2 cm leaf pieces, and the front and back of each leaf piece were sprayed under an Airbrush spray treatment pressure of 10 psi (approximate to 0.7 kg/cm²) and with a spray volume of 0.5 mL. After drying in the shade, 10 3rd-instar larvae were inoculated for each treatment, and each treatment was repeated for 3 times. After the treatment, the leaf pieces were put into an observation chamber at 25° C. and with a relative humidity of 60% to 70%, and the number of living larvae was investigated 3 days after the administration of the compounds, and the death rates were calculated.

Partial test results for Mythimna separata were as follows:

At a dosage of 500 mg/L, 3 days after the administration of the compounds, the death rates of the compounds 1.22, 1.23, and 1.24 for Mythimna separata were above 90%, and the death rates of the control compounds KC1, KC2, and KC3 for Mythimna separata were 0.

(2) Determination of Activity Against Plutella xylostella

Determination Method: cabbage leaves were formed into leaf discs having a diameter of 2 cm using a puncher, and the front and back of each leaf disc were sprayed under an Airbrush spray treatment pressure of 10 psi (approximate to 0.7 kg/cm²) and with a spray volume of 0.5 mL. After drying in the shade, 10 3rd-instar larvae were inoculated for each treatment, and each treatment was repeated for 3 times. After the treatment, the leaf discs were placed into an observation chamber at 25° C. and with a relative humidity of 60% to 70%, and the number of living larvae was investigated 3 days after the administration of the compounds, and the death rates were calculated.

Partial test results for Plutella xylostella were as follows:

At a dosage of 500 mg/L, 3 days after the administration of the compounds, the death rates of the compounds 1.22, 1.23, and 1.24 for Plutella xylostella were above 90%, and the death rates of the control compounds KC1, KC2, and KC3 for Plutella xylostella were 0.

### Example 4: Acaricidal Activity Determination

Greenhouse acaricidal activity determination was carried out on the compounds of the present invention. A determination method was described below.

Each compound to be determined was dissolved in acetone or dimethyl sulfoxide according to the solubility thereof, and prepared into 50 mL of a solution to be determined at a desired concentration with a tween-80 solution of 0.1%, where a content of the acetone or dimethyl sulfoxide in the solution did not exceed 10%.

Two true leaves of Phaseolus vulgaris seedlings were inoculated with adult Tetranychus cinnabarinus and a basic number was investigated. An entire seedling was then sprayed using a hand-held sprayer. Each treatment was repeated for 3 times. After the treatment, the seedlings were placed into a standard observation chamber, and the number of living mites was investigated 72 hours later, and the death rates were calculated.

Test results were as follows:

When the concentration of the solution was 5 mg/L, the death rates of the compounds 1.22, 1.23, and 1.24 for Tetranychus cinnabarinus were above 90%.

Parallel comparison experiments on acaricidal activity were conducted for Tetranychus cinnabarinus using the compounds 1.22, 1.23, and 1.24 and control compounds (3 days after the administration of the compounds), and the determination method was as described above; and the results were shown in Table 14:

TABLE 14
Parallel comparison experiments on acaricidal activity
for Tetranychus cinnabarinus using the compounds
1.22, 1.23, and 1.24 and control compounds

| | |
|---|---|
| | Death Rate (%, 3 days after the |
| Compound | administration of the compounds) |

| No. | 2.5 mg/L | 1.25 mg/L |
|---|---|---|
| 1.22 | 92.4 | 87.7 |
| KC1 | 52.0 | 22.9 |
| 1.23 | 93.8 | 43.9 |
| KC2 | 50.0 | 0 |
| 1.24 | 80.0 | 20.2 |
| KC3 | 0 | 0 |

By a comparison between the compound 1.22 of the present invention and the control compound KC1, a comparison between the compound 1.23 of the present invention and the control compound KC2, and a comparison between the compound 1.24 of the present invention and the control compound KC3, it could be seen that the compounds of the present invention have higher acaricidal activity than those in the prior art.

The inventors of the present invention conducted a lot of experiments, and replaced the methyl on the biphenyl structure in the prior art with the chlorine atom on the basis of the molecular skeletons of existing compounds, thus obtaining the compound of Formula I of the present invention. As can be seen from the activity comparison experiments of Example 3 and Example 4, compared with the prior art, the compound of the present invention have unexpected better insecticidal and acaricidal activities.

In an organic molecule, due to different electronegativeties, volumes, or spatial configurations of substituents, the entire molecule may differ greatly in conduction performance or receptor-binding in organisms such as insects, mites, and plants, and may also exhibit a great bioactivity difference, and the conduction performance and the receptor-binding suitability of the molecule are unpredictable and can be known with a lot of creative efforts. Therefore, the present invention possesses prominent substantive features and represents a notable progress.

1. A biphenyl compound containing substituted sulfide (sulfoxide) groups, shown in Formula I: 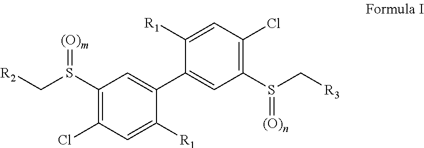
wherein:
R₁ is selected from hydrogen or a halogen;
R₂ and R₃ are each independently selected from hydrogen, cyano, cyclopropyl, C₁-C₃ alkyl, or C₁-C₃ haloalkyl;
m and n are each independently selected from 0 or 1.

2. The compound according to claim 1, wherein in Formula I,
R₁ is selected from hydrogen, fluorine, chlorine, or bromine;
R₂ and R₃ are each independently selected from hydrogen, cyano, cyclopropyl, methyl, ethyl, n-propyl, isopropyl, —CF₃, —CHF₂, —CH₂F, —CH₂CF₃, —CH₂CHF₂, or —CH₂CH₂F;
m and n are each independently selected from 0 or 1.

3. The compound according to claim 2, wherein in Formula I,
R₁ is selected from fluorine;
R₂ and R₃ are each independently selected from hydrogen, cyano, cyclopropyl, methyl, ethyl, n-propyl, isopropyl, —CF₃, —CHF₂, —CH₂F, —CH₂CF₃, —CH₂CHF₂, or —CH₂CH₂F;
m and n are each independently selected from 0 or 1.

4. The compound according to claim 3, wherein in Formula I,
R₁ is selected from fluorine;
R₂ and R₃ are selected from —CF₃;
m and n are each independently selected from 0 or 1.

5. A compound, being an intermediate for preparing the biphenyl compound containing substituted sulfide (sulfoxide) groups according to claim 1, wherein the compound has a structure shown in Formula II: 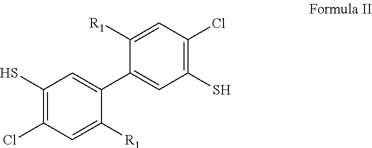
wherein
R₁ is selected from hydrogen or a halogen.

6. An insecticide in the fields of agriculture, forestry, and health, which is the compound of Formula I according to claim 1.

7. An acaricide in the fields of agriculture, forestry, and health, which is the compound of Formula I according to claim 1.

8. An insecticidal and acaricidal composition, comprising the compound of Formula I according to claim 1 and an agriculturally acceptable carrier, wherein the active component in the composition accounts for 0.1-99% by weight.

9. A method for controlling agricultural or forestry pests and pest mites, comprising applying an effective amount of the composition according to claim 8 to pests and pest mites in need of control or a growth medium thereof.
